(19) United States
(12) Patent Application Publication
(10) Pub. No.: US 2001/0016721 A1
(43) Pub. Date: Aug. 23, 2001

(54) Absorbent article having compliant cuffs

(76) Inventors: Catherine E. Salerno, Millington, NJ (US); Tenny Jerschkow, Middletown, NJ (US); Barbara Lemli, Kendall Park, NJ (US); Sherilyn McCoy, Skillman, NJ (US)

Correspondence Address: Philip S. Johnson, Esq., Johnson & Johnson, One Johnson & Johnson Plaza, New Brunswick, NJ, 08933-7003, US

(21) Appl. No.: 09/737,078

(22) Filed: Dec. 14, 2000

Related U.S. Application Data
(63) Continuation-in-part of application No. 09/108,483, filed on Jul. 1, 1998, now abandoned.
Application No. 09/108,483 is a continuation-in-part of application No. 08/522,876, filed on Sep. 1, 1995, now abandoned.

(51) Int. Cl. A61F 13/20

(52) U.S. Cl. 604/386; 604/398

(57) ABSTRACT

An absorbent article is provided for use in the perineal area of the body. Compliant sealing cuffs are pre-formed to extend outward from the central portion of the article. The cuffs may be formed by looping a strip of resilient highloft material covered with a porous material so as to form a compliant cuff which bears against the user's body in a comfortable manner. An elastic member placed in tension so as to impart an arcuate shape to the article may be placed inside a cavity formed by the cuff. The cuffs may be applied to an article having wings attached to the central portion so as to form pockets for retaining the elasticized portions of the panty crotch. The proximal ends of the cuffs are disposed outward of the pockets and the distance between the pockets is less than the width of the panty crotch so that the body faceable side of the article is placed in tension.

This application is a continuation-in-part of application, Ser. No. 09/108,483, now abandoned which is a Continuation-In-Part of application, Ser. No. 08/522,876, now abandoned.

### FIELD OF THE INVENTION

The present invention relates an absorbent article for use in the perineal area of the body, such as sanitary napkins, incontinence pads, and the like, and more specifically relates to an absorbent article having compliant side cuffs along the lateral edges of the absorbent article that provide enhanced conformability of the absorbent article to the wearer's body, thereby inhibiting or preventing leakage past the lateral sides of the absorbent article.

### BACKGROUND OF THE INVENTION

Traditional absorbent articles are generally characterized as having a body fluid pervious material defining a body faceable side, a body fluid impervious material defining a garment faceable side, and a central absorbent core between the body faceable side and the garment faceable side. The body faceable side layer and the garment faceable side layer are generally sealed around the outer edge margins of the central absorbent core so as to form laterally extending flanges (see, for example U.S. Pat. No. 4,678,527 to Ulman).

Under certain circumstances, absorbent articles are subject to lateral leakage, for example, if the article is locally not in contact with the perineum because of wrinkling or deformation of the absorbent article or if the flow of body fluids exceeds the local absorbent capacity of the absorbent article. Such lateral leakage causes fluid to flow along and beyond the surface of the perineum to the user's legs often resulting in soiling of the undergarment or other clothing articles. Attempts have been made to enhance the fit of such absorbent products by imparting an arcuate shape to the article in the longitudinal direction. This is typically accomplished by applying longitudinally extending elastic elements placed in tension to the article (see, for example U.S. Pat. No. 3,236,238 to Morse and U.S. Pat. No. 4,432,823 to Moore).

U.S. Pat. No. 4,701,177 to Ellis et al. discloses a sanitary napkin in which the absorbent core in the central portion of the napkin has a dog bone shape, i.e., the width of the absorbent core is reduced in the central portion. The cover layer and barrier layer are sealed along their lateral edges in a contour which follows the top edges of the lateral sides of the central absorbent core. As a result, in the reduced width central portion of the napkin, there are portions of the cover and barrier which are outward of the absorbent core which tend to form walls. Elastic members disposed within the walls cause them to extend upward above the body faceable surface and into the crease at the sides of the pudendum so as to prevent leakage. However, this arrangement suffers from several drawbacks.

First, as a result of the placement of the seal lines at the top of the sides of the central portion, the walls have a tendency to fold inward about the joint lines in use so that they lay over the body faceable surface, thereby reducing the effective area of the central absorbent portion. Second, the formation of walls requires that the central portion be formed into a dog bone shape so that this sealing approach is not applicable to all types of napkins. Third, the materials that form the walls are limited to those suitable for napkin covers and barriers. Finally, since the perineal area of the body does not have an arcuate or cup shape, these products are not fully effective for maintaining contact between the perineal area and the absorbent product.

Other attempts to prevent lateral side leakage in absorbent articles have been to provide wings or flaps which wrap around the crotch portion of the wearer's undergarment to cover and thus protect the undergarment. For example, U.S. Pat. No. 4,285,343 to McNair or U.S. Pat. No. 4,589,876 to Van Tilburg disclose sanitary napkins having wings in which flexible axes are formed that allow the wings to be folded over the edges of the panty crotch. In Van Tilburg, each wing is joined to the central portion of the napkin along a preferential bending line. The width of the central portion is less than the span of the perineum so that the elastic in the panty crotch bends the wings upward around the preferential bending line. This bending action causes the wings to form walls that bear against the laterally outward surfaces of the perineum to produce a seal that is described as being gasket-like. Unfortunately, such articles suffer from several drawbacks.

First, since the article relies on the panty elastic to bend the wings upward around the laterally outward surfaces of the perineum at the preferential bending joints between the wings and the central portion, the maximum width of the central portion is limited to the width of the panty crotch. This limits the absorbent capacity of the napkin as well as its applicability to a large variety of panty sizes.

Second, since the wings are folded over the edges of the panty crotch, the seals formed thereby can extend beyond the edges of the crotch only by the thickness of the wings. As a result, optimal contact of the seal with the body will not be attained for all users since the seal does not extend a substantial distance beyond the edge of the crotch. Again, this limits the applicability of the napkin.

Third, although the preferential bending line and flexible axis give the wing flexibility in the direction normal to the plane of the wing, the wing is relatively stiff in response to a compression force applied in the plane of the wing. Hence, the compliancy of the wings is low, resulting in discomfort due to the wings digging into the body.

Fourth, since the elastic portion of the crotch is disposed at the top of the wall formed by the wing, the force imposed by deformation of the elastic portion acts to press only the wing against the user's body. The elastic portion does not push the central portion of the article against the perineum so as to ensure proper contact.

Absorbent articles in which the layers are joined by forming flanges, as discussed above, suffer from the drawback that although the flanges are flexible with respect to forces acting perpendicular to the plane of the flange, they are fairly rigid in response to forces acting in plane. Consequently, the flanges of such articles have a tendency to dig into the skin of the user, causing discomfort.

U.S. Pat. No. 4,695,278 to Lawson discloses a diaper in which flaps are formed by extending the cover and barrier beyond the sides of the central portion and joining them together along longitudinally extending joint lines spaced transversely from the central portion sides. Elastic members are disposed within the flaps, to form members characterized as “barrier cuffs.” Members characterized as barrier cuffs are formed by attaching strips of material, folded over so as to form loops at their distal ends, to the flaps along the joint lines. Elastic members are disposed within the loops causing them to extend vertically upward above the body faceable surface. Unfortunately, as a result of the length of the barrier cuffs and the spacing of the joint lines away from the sides of the central portion, the barrier cuffs, like the walls in the Ellis patent, have a tendency in use to fold inward about the joint lines so that they lay over the body faceable surface, thereby reducing the effective area of the central absorbent portion.

Consequently, it is desirable to provide an absorbent article that overcomes the aforementioned drawbacks associated with absorbent articles heretofore known in the art. Such an article should be capable of preventing lateral leakage of fluid; maintain body contact with the effective area of the central absorbent; be adapted to fit properly regardless of the size of the user or the undergarment; be sufficiently compliant to provide a comfortable fit and have the surface properties which minimize any unpleasant sensation of such contact while ensuring a good fit and proper contact of the central portion of the absorbent article with the body.

### SUMMARY OF THE INVENTION

It is an object of the current invention to provide an absorbent article which inhibits or prevents leakage of fluid past the lateral edges of the absorbent article.

It is another object of the current invention to provide an absorbent article which is suitable for use in undergarments having a large range of sizes.

It is yet another object to provide an absorbent article having compliant side cuffs which make good sealing contact with the user's body and yet are comfortable.

In accordance with the present invention, these and other objects have been provided in an absorbent article for use in the perineal area of a user's body to absorb body fluid, said absorbent article having right and left lateral sides and first and second transverse ends, said absorbent article comprising:

a) a fluid pervious first layer forming a body faceable surface;

b) a fluid impervious second layer forming a garment faceable surface opposite the body faceable surface;

c) an absorbent core positioned between the first layer and the second layer;

d) right and left longitudinally extending cuffs, the cuffs having a base portion and a distal end, each of the cuffs comprising a longitudinal strip of a resilient, highloft, fluid permeable material which is covered, at least in part, with a flexible, fluid repellent porous material, and wherein the right and left longitudinally extending cuffs are attached along their respective base portions to the right and left lateral sides of the absorbent article, respectively, such that the distal ends of the cuffs extend outward from the right and left lateral sides of the absorbent article.

Also provided in accordance with the present invention is an absorbent article for absorbing fluid in the perineal area of the user's body and adapted for use in conjunction with an undergarment having a crotch portion having right and left edges, the absorbent article comprising:

a) a fluid pervious first layer forming a body faceable surface, a fluid impervious second layer forming a garment faceable surface opposite the body faceable surface, an absorbent core between the first and second layers and having right and left longitudinally extending opposing sides, the first and second layers having first joining means for joining the first and second layers, thereby forming right and left flanges adjacent the right and left sides of the absorbent core, respectively;

b) right and left longitudinally extending cuffs, each cuff comprising a longitudinal strip of a resilient, highloft, fluid permeable material which is covered, at least in part, with a flexible, fluid repellent porous material, each cuff having a base portion and a distal end, wherein each cuff is attached along its respective base portion to the absorbent article adjacent the right and left flanges and wherein the distal end of each cuff extends outward therefrom, whereby the distal ends of the cuffs are compressed against a portion of the user's body in use;

c) right and left wings each having a base portion and a distal end portion, the base portion being attached to the fluid impervious layer inwardly from the lateral edges of the absorbent article, thereby forming right and left pockets, the distal end portion adapted to fold over and retain the right and left edges of the undergarment crotch portion in the right and left pockets, respectively.

Also provided in accordance with the present invention is an absorbent article for use in the perineal area of a user's body to absorb body fluid, the absorbent article having right and left lateral sides and first and second transverse ends, the absorbent article comprising:

a) a fluid pervious first layer forming a body faceable surface;

b) a fluid impervious second layer forming a garment faceable surface opposite the body faceable surface;

c) an absorbent core positioned between the first layer and the second layer wherein the first layer has right and left approximately longitudinally extending edges, and the second layer has right and left approximately longitudinally extending edges, and wherein the first layer is joined to the second layer adjacent to their respective right and left edges so as to form right and left flanges, wherein the right and left flanges are adjacent the right and left lateral sides of the absorbent article;

d) right and left longitudinally extending cuffs, the cuffs having a base portion and a distal end, each of the cuffs comprising a longitudinal strip of a resilient, fluid permeable, highloft material, and a longitudinal strip of an apertured polymeric film which encloses the highloft material along at least a substantial portion of its length and wherein the right and left longitudinally extending cuffs are adhered along their respective base portions to the right and left lateral sides of the absorbent article, respectively, such that the distal ends of the cuffs extend outward from the right and left flanges;

e) right and left wings each having a base portion and a distal end portion, the right and left wings attached at their respective base portions to the right and left flanges, respectively, and forming a part thereof, and the distal ends being foldable over a crotch portion of a user's undergarment.

### BRIEF DESCRIPTION OF THE DRAWINGS

FIG. 1 is an isometric view of one embodiment of an absorbent article according to the current invention.

FIG. 2 is a plan view of the article shown in FIG. 1.

FIG. 3 is an elevation view of the article shown in FIG. 1.

FIG. 4 is a transverse cross-section through the article shown in FIG. 1 taken through line IV-IV.

FIG. 5 is a transverse cross-section through the article shown in FIG. 1 taken through line V-V.

FIGS. 6(a), 6(b), 7(a) and 7(b) are detailed views of alternative embodiments of the region of the article shown in FIG. 5 enclosed by the circle VI.

FIG. 8 is a transverse cross-section through the embodiment of the article shown in FIG. 1 in use.

FIG. 9 is a transverse cross-section through another embodiment of the article.

FIG. 10 is a transverse cross-section through a still another embodiment of the article.

FIGS. 11(a)-(d) are detailed views of embodiments of the portion of the article shown in FIG. 10 enclosed by the circle X.

FIG. 12 is a transverse cross-section through a still another embodiment of the article.

FIGS. 13(a)-(c) are detailed views of embodiments of the portion of the article shown in FIG. 12 enclosed by the circle XIII.

FIG. 14 is an isometric view of yet another embodiment of an absorbent article according to the current invention having wings.

FIG. 15 is an elevation view of the article shown in FIG. 14.

FIG. 16 is a transverse cross-section through the article shown in FIG. 14.

FIGS. 17(a)-(c) are detailed views embodiments of the portion of the article shown in FIG. 16 enclosed by the circle XVII.

FIG. 18 is a transverse cross-section through the article shown in FIG. 14 in use.

FIG. 19 is a plane view from below of the embodiment of the article.

FIG. 20 is a transverse cross-section through another embodiment of the article shown in FIG. 14 in use.

FIG. 21 shows an alternate embodiment of the cuff.

FIG. 22a shows an alternate embodiment of the cuff.

FIG. 22b shows another alternate embodiment of the cuff.

### DESCRIPTION OF THE PREFERRED EMBODIMENTS

The present invention is directed to an absorbent article for use in the perineal area of a user's body to absorb body fluid. The absorbent article has right and left lateral sides and first and second transverse ends and comprises: a) a fluid pervious first layer forming a body faceable surface; b) a fluid impervious second layer forming a second surface opposite the body faceable surface; c) an absorbent core positioned between the first layer and the second layer; d) right and left longitudinally extending compliant cuffs.

The cuffs comprise a longitudinal strip of high loft material and a longitudinal strip of fluid repellent porous material which encloses the high loft material along at least a substantial portion of its length. That is, the cuff comprises an inner layer which comprises a high loft material and an outer layer which comprises a fluid repellent porous material. The compliant cuffs of the present invention have a base portion and a distal end portion, and are attached along a substantial portion of their respective base portions to the right and left lateral sides of the absorbent article, respectively, such that the distal end portions of the cuffs extend outward from the right and left lateral sides of the absorbent article.

The strip of high loft material 47 may be formed from a fibrous woven or nonwoven flexible fabric that is soft, comfortable, and cushiony, to provide a comfortable “feel” to the user, and also possesses an open, fluid-permeable structure, i.e., having void spaces which are capable of holding or retaining fluid, and which may optionally be capable of drawing fluid away from the fluid repellent porous material which covers the high loft material to provide a ‘clean-dry’ appearance to the cuff. As used herein the term ‘high loft material’ refers to materials which either as a single layer or as a multi-layer laminate provide a total thickness of the cuff of at least 25 mils. The exact thickness of either the high loft material or the fluid repellant porous material is not per se critical to the invention, provided of course, that the total thickness of the cuff is at least 25 mils, preferably at least 40 mils, and most preferably between 40 and 80 mils. The strip of high loft material 47 may be wicking or non-wicking, and is preferably non-wicking so as not to promote the flow of fluid beyond the cuff 6.

The resilient, fluid permeable high loft material is preferably flexure resistant. Flexure resistance is generally measured by peak bending stiffness which is more fully discussed in U.S. Pat. No. 5,171,302 to Buell, which is incorporated herein in its entirety. The high loft material of the present invention preferably has a peak bending stiffness of at least 100 grams, preferably greater than 200 grams, and most preferably between 250 grams and 1000 grams.

The resilient, highloft, fluid permeable material may be formed from fibers selected from the group consisting of polyester fibers, polyethylene/polyester bicomponent fibers, polyethylene/polypropylene bicomponent fibers, polypropylene/polyester bicomponent fibers, high melting/low melting polyester bicomponent fibers, air laid pulp, pulp-fiber blends, and combinations thereof. The resilient, fluid permeable high loft materials may alternatively be formed from a polymeric foam. Laminate structures formed from the above materials is also considered to be within the scope of the present invention. However, it is important that the upper-most layer in the laminate structure, (which corresponds to the body-faceable side of the absorbent article) possesses an open, fluid permeable structure capable of holding or retaining fluid, and combinations of the above materials. The cuffs may further comprise a layer of a perforate or non-perforate flexible material which is laminated to the strip of resilient, fluid permeable, highloft material to form a laminated structure, with the proviso that at least the top layer of the laminated structure comprises a fluid permeable material. The strip of resilient, fluid permeable, highloft material and/or the layer of perforate or non-perforate flexible material may be elastic. When the high loft material is elastic, it is applied to the cuff in tension, the tension being in an amount sufficient to form the absorbent article into an arcuate shape in the longitudinal direction. A particularly preferred resilient, fluid permeable, high loft material is a fusible fiber which is commercially available under the trademark ENKA having a bulk density of about 0.5 oz./sq. yd. and a thickness of about 9 mils.

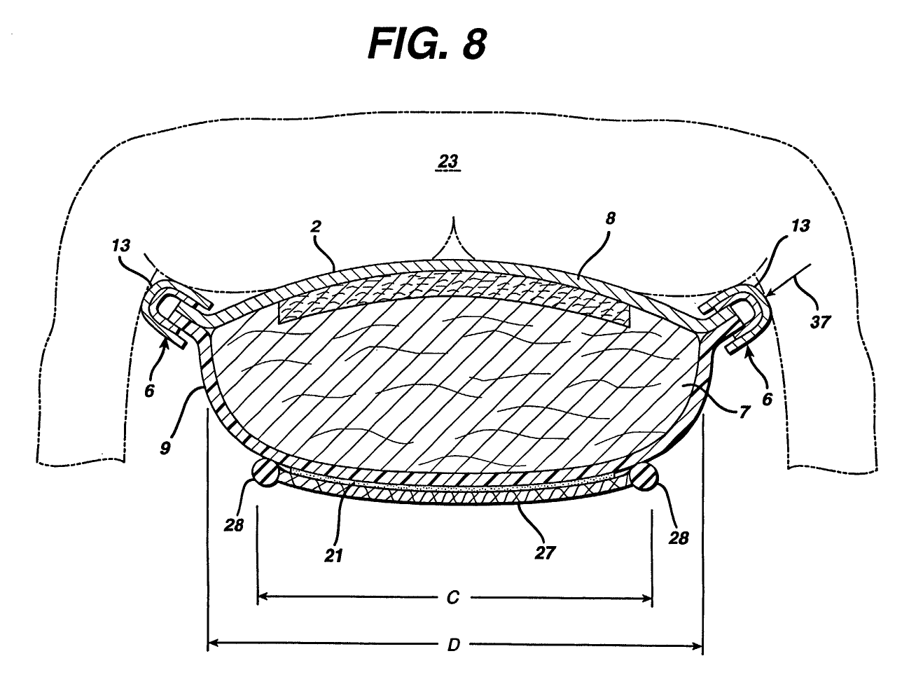

In addition to a comfortable feel, the cuffs preferably provide a smooth, curved, edgeless surface at their distal ends that serves as the contact surface for the cuff against the body, as shown in FIG. 8. Such contouring of the cuff contact surface minimizes any discomfort associated with such contact under pressure.

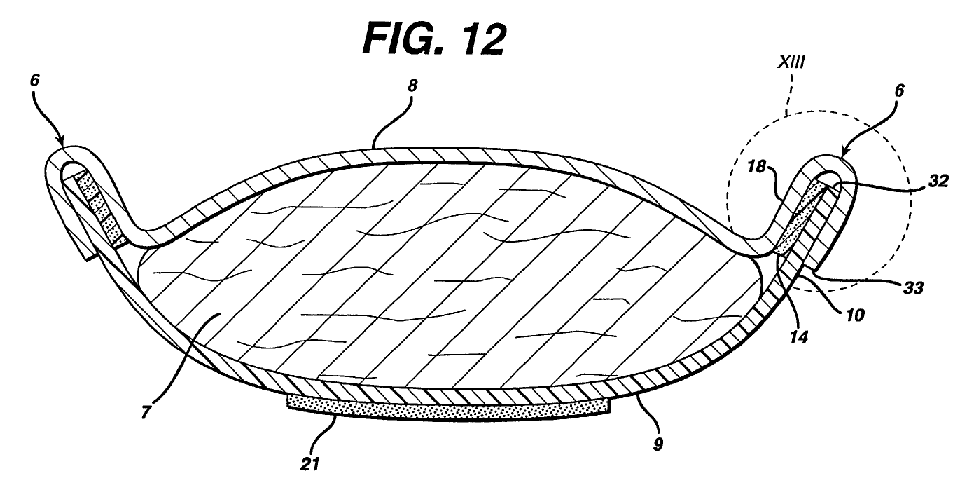

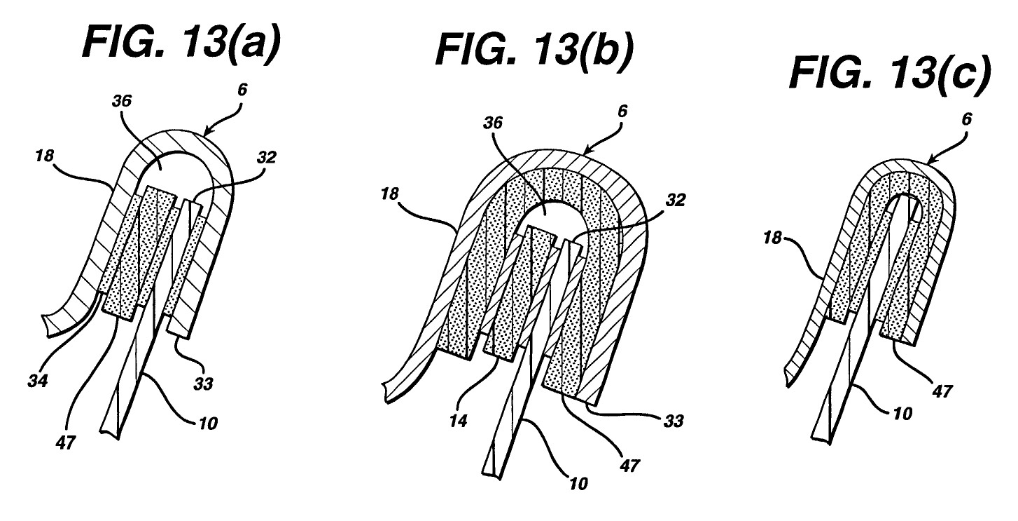

Suitable materials for use as the fluid repellent porous material include, but are not limited to, any of the conventional fluid repellent materials used as fluid pervious cover layers in commercially available absorbent sanitary products such as apertured polymeric films, nonwoven fabrics and woven fabrics. A preferred material for use as the fluid repellent porous material is a hydrophobic apertured polymeric film. In a most preferred embodiment, the apertured polymeric film is applied as a separate strip of material which is attached to the side cuffs while the film is tensioned in an amount sufficient to form the absorbent article into an arcuate shape along a longitudinal dimension of the article. Alternatively, it is also possible to use the same apertured polymeric film which was used as the cover layer to simultaneously form the side cuffs as shown in FIGS. 12-13. In another embodiment of the invention, each of the cuffs comprises a laminated structure having a top layer and a bottom layer, wherein one or more layers of polymeric film or polymeric foam material are laminated to one or more layers of the resilient, fluid permeable, highloft material, with the proviso that the top layer of the laminated structure comprises a fluid permeable material. Optionally, one of the layers of polymeric film or polymeric foam material is tensioned prior to being laminated to the one or more layers of the resilient, fluid permeable, highloft material.

The surface 16 of the absorbent article that is intended to be worn against the body of the user (i.e. the body faceable surface) is covered by a layer 8 of a body-fluid pervious material, typically referred to as a “cover layer”. The cover layer 8 may be formed from any fluid pervious material that is comfortable against the skin and that permits fluid to penetrate to the underlying core 7, which retains the fluid. The cover layer 8 is preferably fluid repellent, i.e. it should retain little or no fluid in its structure to provide a relatively dry surface next to the skin. The fluid pervious cover layer 8 may be a fibrous woven or nonwoven fabric made of fibers or filaments of polymers such as cotton, rayon, nylon, polyethylene, polypropylene, polyester, bi-component fibers of polyester sheathed in polyethylene or polypropylene, bi-component fibers of polypropylene sheathed in polyethylene, fibers of high melting polyester sheathed in low melting polyester, and combinations thereof.

In a most preferred embodiment, the cover layer 8 may be formed from an apertured polymeric film. The thickness of the cover layer 8 will vary from approximately 0.001 to 0.062 inch, preferably from 0.005 to 0.020 inch and most preferably from 0.010 to 0.015 inch. Generally, the fluid pervious cover layer 8 is a single sheet of material having a surface area sufficient to cover the body-faceable surface 16 of the absorbent article. Preferably, the fluid pervious cover layer 8 is longer than the core 7 so as to form the transverse ends 3. The transverse ends 3 may be sealed with other pervious or non-pervious layers to fully enclose the central absorbent core.

The absorbent article 1 further comprises a layer 9 of a body fluid impervious material, typically referred to as a “barrier layer”, on its garment faceable surface 17. The fluid impervious barrier layer 9 may comprise any thin, flexible, body fluid impermeable material and is typically a polymeric film such as, for example, polyethylene, polypropylene, cellophane, and the like. Alternatively, the barrier layer may be a normally fluid pervious material that has been treated to be impervious, such as impregnated fluid repellent paper, woven or nonwoven fabric material, closed cell flexible foams, such as polyurethane or cross-linked polyethylene. The fluid impervious barrier layer may optionally be permeable to gas including water vapor to provide an air breathable fluid barrier. The thickness of the barrier layer is not per se critical to the invention, provided of course that it is flexible. A typical barrier layer thickness, when formed from a polymeric film, is in the range of from 0.0005 to 0.025 inch and is preferably in the range 0.001 to 0.002 inch.

The barrier layer 9 generally comprises a single sheet of material having a surface area sufficient to cover the entire garment-faceable surface 17 of the absorbent article. The fluid impervious barrier layer 9 may extend around the sides of the core 7 in a C-shaped configuration (not shown) with the portions 10 of the barrier layer that are adjacent its longitudinal edges 32 extending upwardly from the garment faceable surface 17 toward the body faceable surface 16 so as to form a portion of the lateral sides 30 of the absorbent article 1.

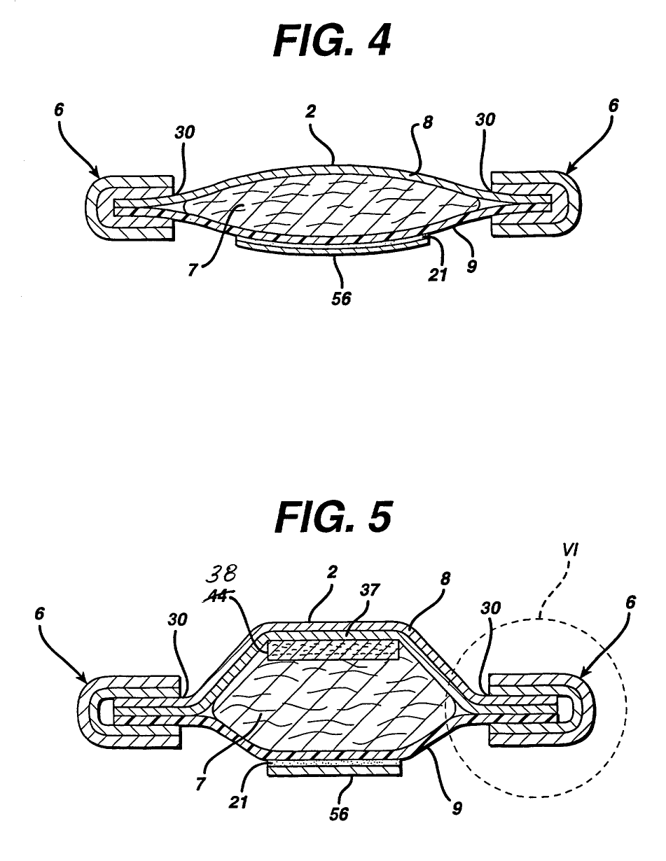

The construction the absorbent article 1 is shown in FIGS. 4 and 5. The central portion 2 of the absorbent article contains an absorbent core 7. As is known in the art, the absorbent core 7 may be comprised of a loosely associated absorbent hydrophilic material such as cellulose fibers, including wood pulp, wet or dry cross-linked wood pulp, curly wood pulp fibers, rayon fibers or cotton fibers, or other absorbent materials generally known in the art, including acrylate fibers, polyvinyl alcohol fibers, peat moss or super-absorbent particles or fibers. The absorbent core may also comprise synthetic fibers such as nylon, polyethylene, polypropylene, polyester, bi-component fibers of polyester sheathed in polyethylene or polypropylene, bi-component fibers of polypropylene sheathed in polyethylene, fibers of high melt polyester sheathed in low melt polyester, and combinations thereof. These fibers may be layered to form an absorbent core having a porosity gradient, and/or may be treated with a surfactant to make them hydrophilic and thereby wettable.

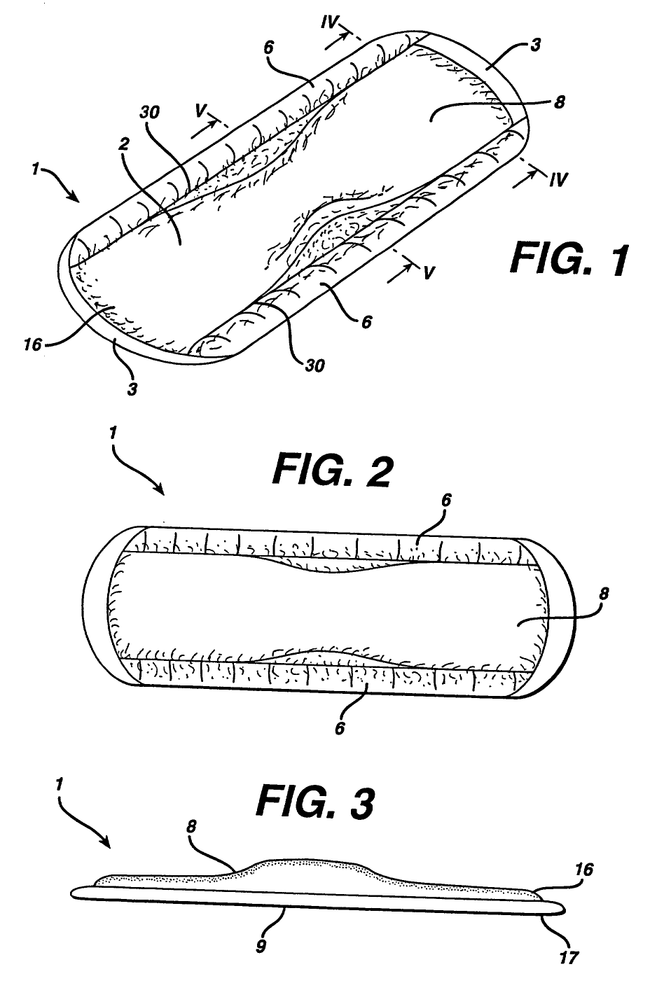

In a preferred embodiment, the central portion of the absorbent core has an hour glass shape and is thicker (i.e. in the “z” direction) in its center region than at its end portions, thus creating an absorbent article having a raised center portion, as shown in FIGS. 1-5. The relative thickness of the center region is not, per se, critical to the invention, and is generally from 1.1 to 4 times thicker than the transverse end regions of the absorbent core, preferably from 1.5 to 3 times thicker, and most preferably from 1.75 to 2.75 times thicker. The raised center portion has been found to provide enhanced body contact with the perineal area of the user's body, and thus enhances the transference of fluid from the user's body into the central absorbent core. In a most preferred embodiment, the raised center portion of the absorbent core further comprises a resilient absorbent structure 38 derived from sphagnum moss. Absorbent structures derived from sphagnum moss are more fully disclosed in U.S. Pat. No. 4,170,515 to Lalancette et al., U.S. Pat. Nos. 4,215,692, 4,226,237, and 4,507,122 to Levesque, U.S. Pat. No. 4,305,393 to Nguyen, U.S. Pat. No. 4,473,440 to Ovens, U.S. Pat. No. 4,618,496 to Brasseur, U.S. Pat. No. 4,676,871 to Cadieux et al., U.S. Pat. No. 4,992,324 to Dube, and U.S. Pat. No. 5,053,029 to Yang which are incorporated by reference herein in their entirety.

The absorbent article of the present invention may optionally include a transfer layer between the fluid pervious cover layer and the absorbent core. The transfer layer generally comprises similar materials as those used in the absorbent core, but will have a more open porous structure than the central absorbent core having interstitial spaces between the fibrous materials which are greater than those in the absorbent core. This creates a porosity gradient which greatly enhances the rate of fluid transfer from the cover layer to the absorbent core which acts as a reservoir for the absorbed fluids.

There is shown in FIGS. 1-3 one embodiment of the current invention illustrated with respect to an absorbent article 1 according to the current invention. The absorbent article is comprised of a longitudinally extending central portion 2 having longitudinal sides 30 and transverse ends 3. The central portion may have an approximately rectangular shape, an approximately oval shape, or preferably an hour-glass or dog-bone shape wherein the transverse ends are wider than the central portion of the absorbent article.

As shown best in FIGS. 1, 4, and 5, right and left hand longitudinally extending cuffs 6 are attached at their bases to the lateral sides 30 of the central portion 2. According to the current invention, the cuffs 6 are in a general longitudinal rectangular configuration so as to form substantially parallel cuffs along the lateral sides of the absorbent article. As shown in FIGS. 1 and 2, the cuffs extend substantially the entire length of the absorbent article. While the length of the cuffs is not, per se, critical to the invention, it is important that the cuffs extend at least across the central portion of the absorbent article, i.e., in the area of the absorbent article which will be adjacent to the perineal area of a user. Accordingly, the length of the cuffs will generally extend at least 50% of the absorbent article length, preferably the cuffs extend at least 75% of the absorbent article length, and most preferably the cuffs extend the entire length of the absorbent article.

There are a number of factors which are considered important in determining the width of the compliant cuffs of the present invention. One factor is that the width of the absorbent article should be at least as wide as the width of the crotch portion of a user's panty. These widths can vary widely and are generally in the range of from about 2.5″ to about 4″. Another factor to consider is the width of the central absorbent core. The central absorbent core should be wide enough to assure a good fit against the perineal area of the user but should not be wider than the perineal area of the user since this may lead to discomfort due to rubbing and/or chafing of the inner thighs of the user. While the anatomy of the perineal area can vary widely, it has generally been found that the central absorbent cores can have widths in the range of from about 2″ to 3″. In consideration of the foregoing factors, the compliant cuffs of the present invention will accordingly have widths in the range of from about 0.25″ to about 1″, preferably in the range of about 0.3″ to about 0.8″, and most preferably about 0.75″.

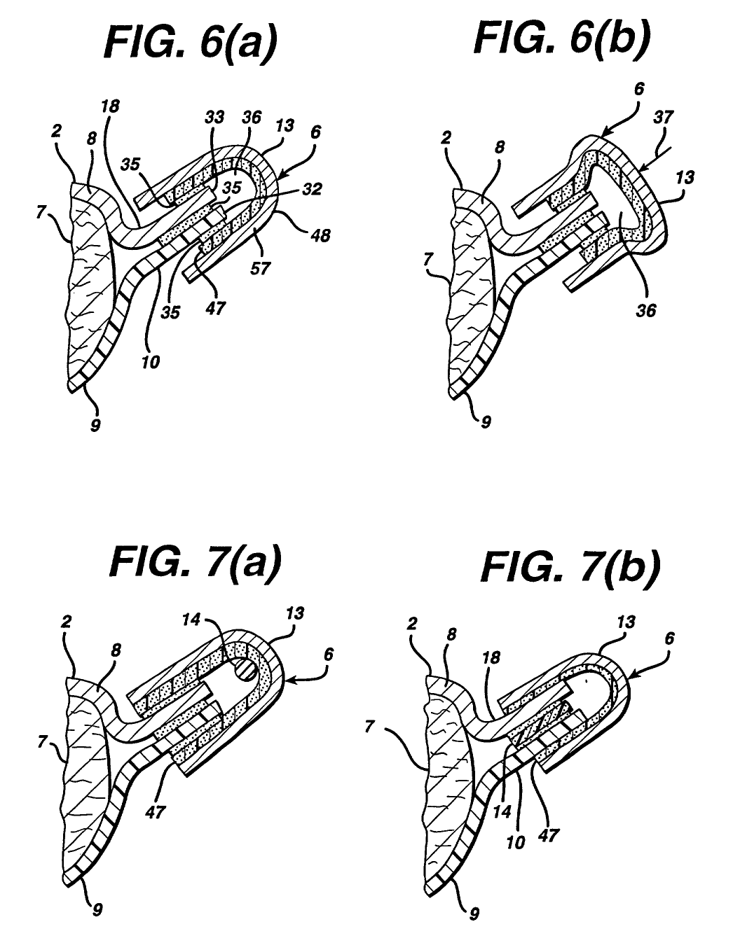

As shown in FIG. 6(a), one embodiment of the invention comprises an absorbent article having side cuffs, wherein each compliant cuff 6 is formed by joining the portions 18 of the cover layer 8 adjacent its longitudinal edges 33 to the portions 10 of the barrier layer 9 adjacent its longitudinal edges 32 via an adhesive 35, thereby forming a flange, and enclosing the flange and the strip of high loft material 47 in a strip of an apertured polymeric film 57 attached thereto via adhesive 35. In the current embodiment, it is considered an important feature of the present invention that the strip of resilient, fluid permeable, high loft material 47 cover at least the upper surface of the flange, and preferably covers both the upper, body-faceable surface of the flange as well as the lower, garment faceable surface of the flange. Covering both surfaces of the flange may be accomplished by adhering two separate strips of high loft material 47 to the outer surfaces of the flange, or by folding a single strip of high loft material around the outer edge margin of the flange, i.e. in a “C” fold configuration.

According to another embodiment of the current invention, the cuff 6 may be attached to the absorbent article, preferably to the flanges of the absorbent article, in a configuration so as to form a loop which encloses a cavity 36, discussed further below. The loop configuration of the cuff 6 provides a compliant gasket effect while the combination of high loft resilient material 48 with fluid repellant porous cover material provides a clean dry appearance to the absorbent article. Thus, the rigidity and directional stability provided by the resilient high loft material in the cuff 6 is not obtained at the detriment of comfort. Specifically, the distal ends 13 of the cuffs are readily deformed by an inward force, imparted by the user's thigh which act in the plane of the cuff, as shown in FIG. 8. The force is elastically absorbed by the flexure resistance of the high loft material in the compliant cuff which permits a bending deformation of the loop which flattens the cavity 36, as shown in FIG. 6(b). In this manner the cuff 6 remains in sealing contact with the body throughout a range of motion due to the resilient, flexure resistant nature of the high loft material. Moreover, the compliancy of the cuff 6 can be varied by adjusting the size and shape of the cavity 36, and the thickness and resilience of the high loft material.

A further advantage of the flattening effect of the cuff 6 is that as the compression forces increase, the cuff geometry automatically adjusts itself so as to increase the area over which the contact force is distributed, thereby further minimizing the awareness of contact.

As shown in FIG. 5, in the longitudinal middle section of the absorbent article having an optional transfer layer 37, between the cover 2 and the absorbent core 7, the cuffs 6 extend outward from the lateral sides of the absorbent article and are preferably maintained within the plane of the base portions of the cuff. That is it is preferred that the cuffs remain substantially flat, i.e. the cuffs preferably remain in a plane which is substantially parallel to the cover layer and/or the barrier layer of the absorbent article. However, the cuffs may optionally extend a distance above the body faceable surface 16 of the central portion 2—specifically, above the portion of the body faceable surface 16 that is adjacent the sides 30 of the central portion. In accordance with this aspect of the current invention, the distance by which the cuffs extend above the adjacent portion of the body faceable surface 16 may be greater than zero to enhance a sealing contact with the user's body, as shown in FIG. 8. However, this distance must not be so great, notwithstanding the aforementioned directional stability, that the cuffs 6 fold inwardly over the body faceable surface 16 in use, thereby covering a substantial portion of the body faceable surface and preventing it from passing fluid to the absorbent core 7.

As shown in FIG. 8, the side edges 28 of the panty crotch 27 need not press directly against the cuffs to extend them into sealing contact with the perineum. As a result, the width D of the central portion 2 can be greater than the width C of the panty crotch 27, as shown in FIG. 8, thereby permitting, if desired, the central portion to be sized to have maximum absorbency yet remain suitable for use with panties of any size.

In another embodiment, the absorbent article 1 is curved in the longitudinal direction so that it has an arcuate shape, thereby better conforming the shape of the absorbent article to that of the body and improving the fit. In accordance with this embodiment of the invention, elastic members may be incorporated into the loop type cuff 6 or alternatively the apertured polymeric film may be applied to the cuffs in tension to create an arcuate shape in the longitudinal direction of the absorbent article.

In one aspect of this embodiment, an elastic element 14, e.g., an elastic polyurethane foam, is laminated to the interior surface of the cuff 6, as shown in FIG. 7(b). In this embodiment, the elastic element 47 extends essentially the length of the cuff 6 and may be attached to the cuff at its ends. The elastic element 14 may be tensioned or untensioned, where, in its untensioned state, the elastic element provides enhanced resilience to maintain the side cuffs ability to retain their original shape after the application of deformation forces from a user's thighs. Alternatively, the elastic foam may be applied to the absorbent article in tension to impart an arcuate shape to the absorbent article. In this embodiment, the elastic element, in its non-deformed state, is shorter than the cuff 6 so that the elastic element is placed in tension by extending it at least 15% when it is attached to the ends of the cuff. When released, the elastic element returns to its approximate original length, thereby forcing the article into an arcuate shape. In yet another alternative embodiment, the length of the elastic member may optionally span only a portion of the cuff length while still being placed in tension so as to impart an arcuate shape. In the preferred embodiment, the elastic element extends at least 30% of the length of the cuff.

The elastic element 14 may be disposed in the cavity 36 formed within the cuff 6 and attached thereto at its ends, as shown in FIG. 7(a) or the elastic foam strip 14 may be disposed between the portions 18 and 10 of the cover layer and barrier layer that form the flange, as shown in FIG. 7(b). Alternatively, the strip of high loft material 47 forming the cuff 6 could itself be a porous elastic foam applied to the absorbent article in tension, as shown in FIG. 7(b).

The arcuate shape could also be imparted by applying to the cuffs heat shrinkable elements—such as filaments formed from vinylidene chloride copolymer microtape, as disclosed in U.S. Pat. No. 3,236,238 to Morse, hereby incorporated by reference in its entirety. Such filaments are heated after application to the cuffs, thereby causing them to shrink so as to impart an arcuate shape to the absorbent article.

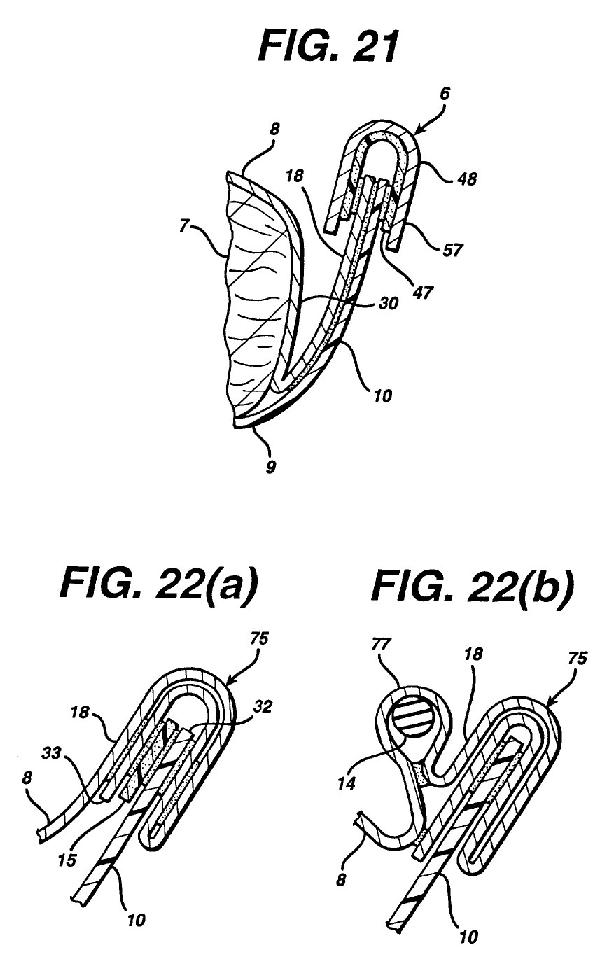

In FIG. 6(a), the flange portion is shown as beginning near the top of the side 30 of the central portion 2. However, as shown in FIG. 21, the flange could be formed by joining the portions 18 and 10 of the cover and base near the bottom of the side 30 and extending the flange upward therefrom to form the cuff.

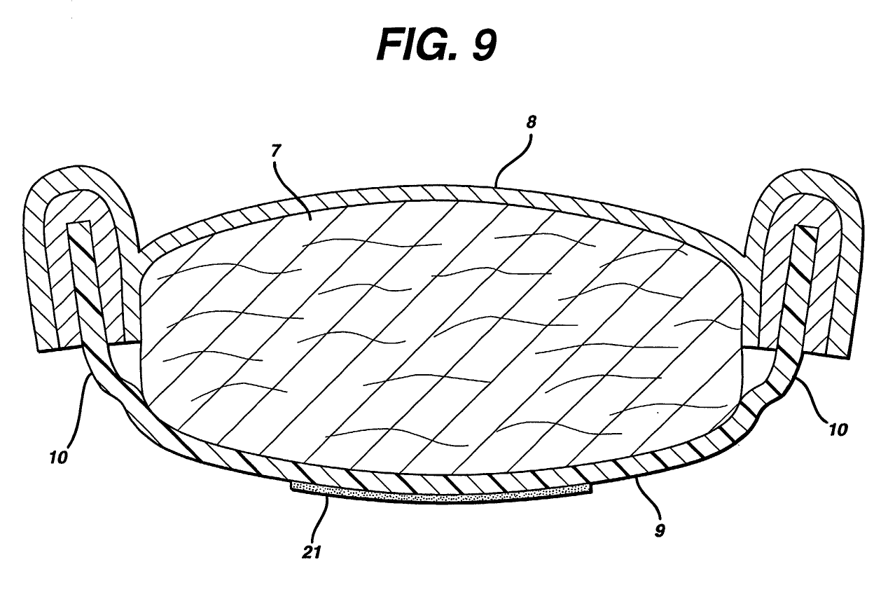

In yet another alternate embodiment of the absorbent article according to the current invention is shown in FIG. 13(b). According to this embodiment, the cuff further contains a substantially flat strip of a flexible resilient material 14 attached to the barrier layer 10. The strip 14 may be formed from a cross-linked foam, such as VOLARA, supplied by Voltek, a division of Sekisui America Corporation of Lawrence, Mass, having a thickness in the range of approximately 0.03 to 0.12 inch. The strip 14 is advantageously disposed between the portion 10 of the barrier layer 10 and the high loft material 47 adjacent their longitudinal edges respectively, and attached via adhesive to each. Moreover, as shown in FIG. 9, the barrier layer portion 9 may be extended so as to cover substantially all of the outward facing surface of the cuff, thereby further preventing leakage.

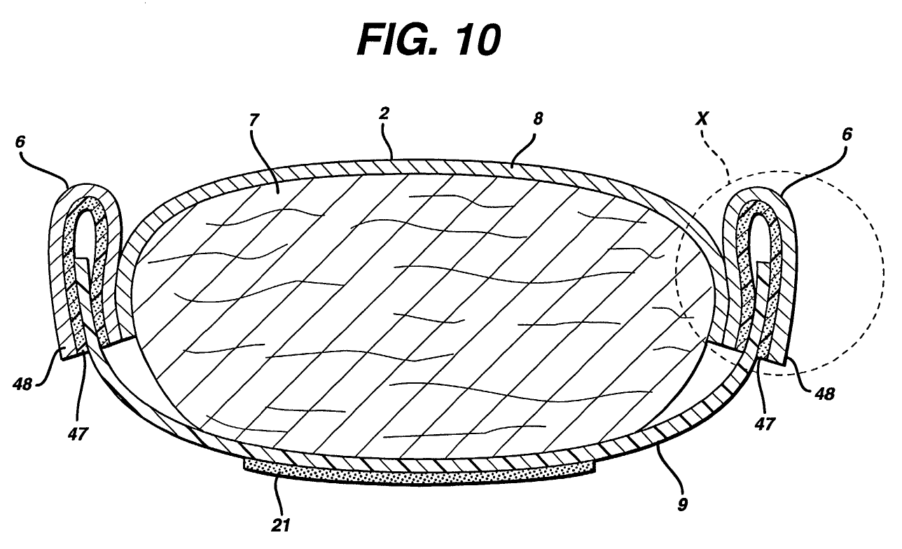

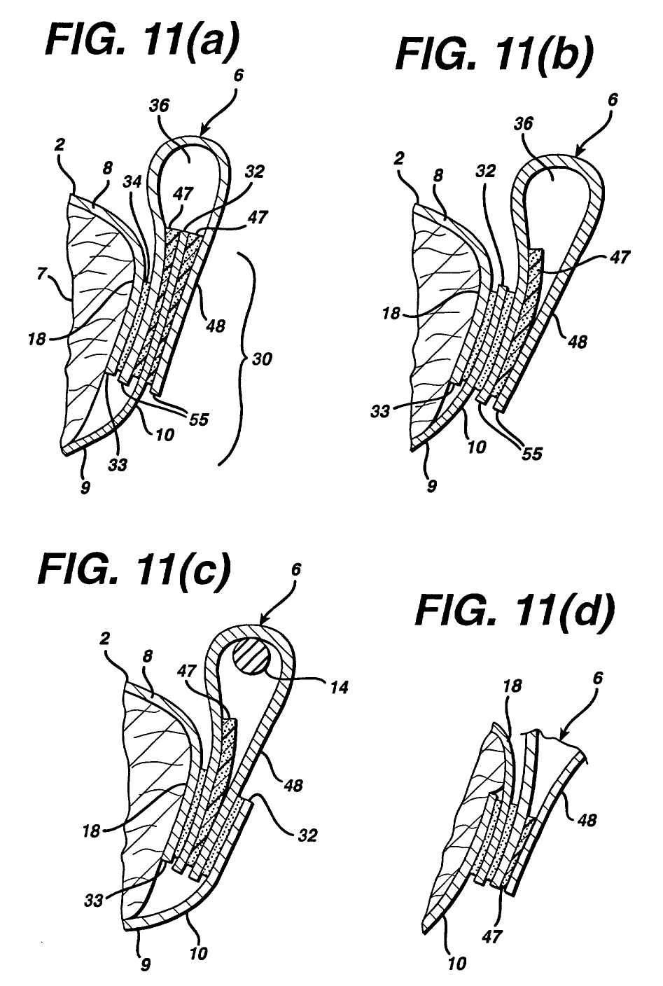

FIGS. 10 and 11(a) show another embodiment of the current invention. In this embodiment, the cuff 6 is formed by bringing together the longitudinal edges of a strip of resilient high loft material 47 covered with an apertured polymeric film so as to form a loop. As shown in FIG. 11(a), the cuff 6 is attached to the sides 30 of the central portion 2 by joining, using adhesive 34, the interior surfaces of the strip 55 adjacent its longitudinal edges to the inward and outward facing surfaces of the portion 10 of the barrier layer 9 adjacent its longitudinal edge 32 so that the loop forms a cuff 6 that encloses the portion of the barrier layer adjacent its longitudinal edge. In addition, the outward facing surface of the portion 18 of the cover layer 8 that is adjacent its longitudinal edge 33 is joined to the inward facing surface of the cuff 6 by adhesive 34.

As shown in FIG. 11(b), the cuff 6 could be formed by joining the interior surfaces of the two portions of the strip of material 55 adjacent its longitudinal edges directly together, and then attaching the inward facing surface of cuff 6 to the outward facing surface of the portion 10 of the barrier layer 9 adjacent its longitudinal edge 32 and attaching the inward facing surface of the portion of the barrier layer adjacent its longitudinal edge to the outward facing surface of the portion 18 of the cover layer 8 adjacent its longitudinal edge 33 so that the portion 10 of the barrier layer 9 is disposed between the cuff and the portion 18 of the cover layer 8. Also, as shown in FIG. 11(c), the outward facing surface of the cuff 6 could be attached to the inward facing surface of the portion 10 of the barrier layer and the inward facing surface of the cuff attached to the outward facing surface of the portion 18 of the cover layer so that the cuff is disposed between the portions 10 and 18 of the barrier layer and cover layer, respectively.

Alternatively, as shown in FIG. 11(d), the cuff 6 could be attached to the outward facing surface of the portion 18 of the cover layer 8 adjacent its longitudinal edge 33 and the inward facing surface of the portion of the cover layer adjacent its longitudinal edge attached to the outward facing surface of the portion 10 of the barrier layer 9 adjacent its longitudinal edge 32 so that the portion 18 of the cover layer 8 adjacent its longitudinal edge 33 is disposed between the cuff and the portion 10 of the barrier layer 9 adjacent its longitudinal edge 32.

Importantly, in each of the approaches to attaching the cuffs to the central portion 2 shown in FIGS. 10 and 11, the cuff 6 is attached to the lateral sides of the absorbent article 30 along a portion of the surfaces forming the sides of the cuff, rather than along its edges. Thus, at least a portion of each of the sides 30 of the central portion is formed from a laminate comprising layers of cuff, cover layer and barrier layer material. Unlike prior art attempts at forming cuffs, the cuffs are not attached along flexible joint lines adjacent the tops of the sides 30 of the central portion or transversely spaced apart from the sides 30, which would allow them to freely bend. Such prior art flexible joints have the undesirable characteristics of requiring the presence of elastic within the cuffs or contact between the elastic in the panty crotch and the cuffs in order to maintain them in the upright position. Such flexible joints also allow the cuffs to fold over the body faceable surface 16 of the central portion, thereby reducing its effective area.

By contrast, the attachment method according to the embodiment of the current invention shown in FIGS. 10 and 11, gives adequate directional stability to the cuffs so that they will extend upward so as to make good sealing contact with the perineum without the incorporation of elastic members into the cuffs. Moreover, provided their width (as measured from the base portion to the distal end) is not excessive, the directional stability of the attachment method according to the current invention will prevent the cuffs from folding over the body faceable surface 16 in use.

As shown in FIG. 11, the loop type cuffs 6 optionally form cavities that impart compliancy to the cuff, as previously discussed with respect to the embodiment shown in FIG. 6(a). Moreover, although, unlike some prior art cuffs, the cuffs according to the current invention do not require the presence of elastic members to cause them to extend upright, elastic members, such as those previously discussed with respect to the embodiment shown in FIGS. 6(a), 6(b), 7(a) and 7(b), may be advantageously incorporated into the loop type cuff 6 to create the arcuate shape. FIG. 11(c) shows a cuff 41 in which an elastic filament 14 is disposed in the cavity 36 FIG. 12 shows still another embodiment of the current invention. In this embodiment, each cuff 6 is formed by extending the cover layer 8 so that the portion 18 adjacent its longitudinal edge 33 is wrapped around the portion 10 of the barrier layer adjacent its longitudinal edge 32, thereby forming a laminated cuff.

FIG. 13(a), 13(b) and 13(c) show three embodiments of the cuffs 6 shown in FIG. 12. As previously discussed, a cavity 36 can be formed inside the cuff so as to impart additional compliancy to the cuff 6. Moreover, a strip of elastic foam 14, placed in tension when applied to the absorbent article, can be disposed within the laminate to impart the aforementioned arcuate shape to the article. Additionally, a layer of porous, high loft foam 47 could be laminated to the inner surface of portion 18 of the cover layer 8 to further increase the compliancy of the cuff, as shown in the embodiment in FIG. 13(b). Alternatively, the porous high loft foam 46 could be laminated to the outer surface of cover layer portion 18. As shown in FIG. 13(c), a strip of elastic foam 47 placed in tension can be wrapped around the barrier layer portion 10 to provide both compliancy and shaping. In the embodiment shown in FIG. 13(c), the cavity 36 has been eliminated, relying entirely on the foam strip 47 for compliancy.

FIG. 22(a) shows another embodiment of the absorbent article shown in FIG. 12, in which the cuff 75 is formed by folding the portion 18 of the cover layer over on itself before using it to enclose the portion 10 of the barrier layer, so that a double layer of the cover layer forms the cuff. As shown in FIG. 22(b), an elastic element, attached to the absorbent article in tension, could be disposed within a secondary loop formed within the folded over portion of the cover layer portion 18 so as to form a secondary cuff 77 in addition to the primary cuff 75.

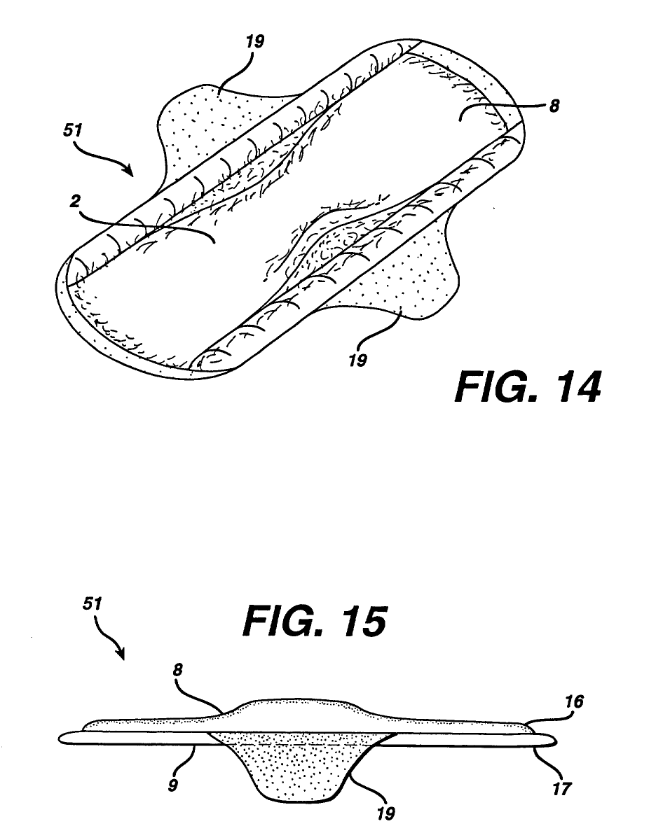

As shown in FIGS. 14 and 15, the current invention may be advantageously adapted for use in a winged absorbent article 51. The wings 19 extend laterally outward from the absorbent article central portion 2. Although preferably not including absorbent pulp materials, the wings 19 may include a body fluid impervious backing such as the materials described in connection with the above-mentioned body fluid impervious barrier layer 9. It is also expected that the wings 19 can comprise a body fluid pervious material, much like the above-mentioned body fluid pervious cover layer 8. According to the current invention, the wings 19 are of the “cut and paste” type—that is, the wings are not integrally cut from the sheets of material forming the cover layer 8 and barrier layer 9 but are formed separately and attached to the central portion 2 via an adhesive. Such cut and paste wings are disclosed in U.S. Pat. No. 4,900,320 (McCoy), hereby incorporated by reference in its entirety. Consequently, the wing material need not be of the type suitable for a pervious cover layer 8 or an impervious barrier layer 9.

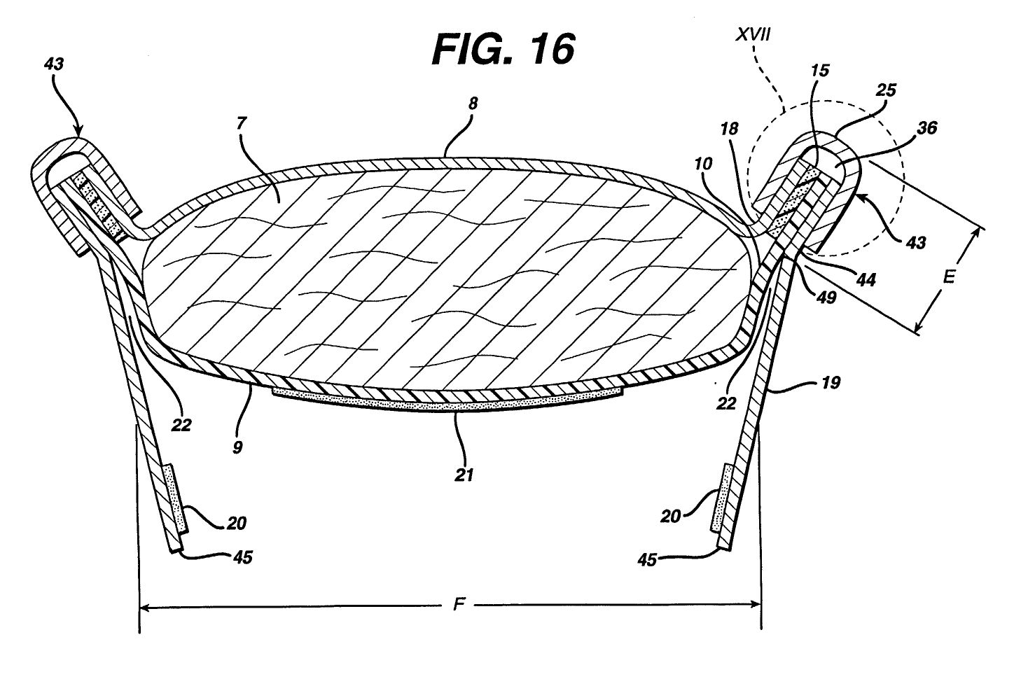

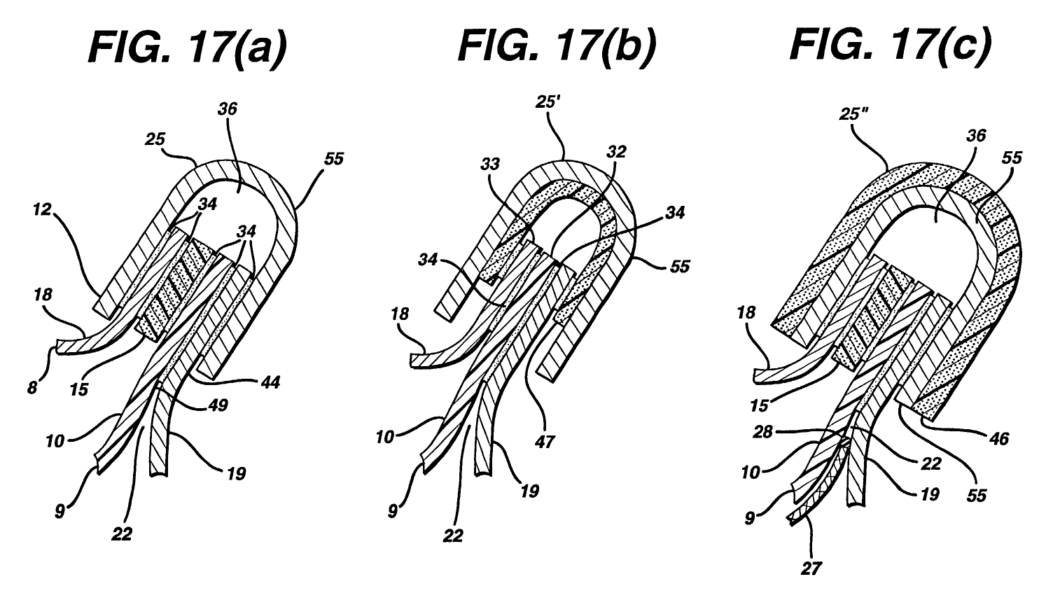

As shown in FIGS. 16 and 17, each cuff 43 may be formed by joining the portions 10 and 18 of the barrier layer 9 and cover layer 10, respectively, adjacent their longitudinal edges together via an adhesive 34, thereby forming a flange. A compliant cuff 25 is formed by enclosing the flange or joined portion in a strip of resilient high loft material 55, such as that used to form the cuffs 6 shown in FIGS. 6(a), 6(b), 7(a) and 7(b). The cuff 25 may be attached so as to form a loop that encloses a cavity 36, as previously discussed, thereby giving it considerable compliancy. As before, the size and shape of the cavity 36 can be adjusted to control the compliancy of the cuff.

A strip of elastic foam 15, placed in tension when applied to the absorbent article, may be disposed between the barrier layer and cover layer portions 10 and 18, respectively, that form the flange so as to impart the aforementioned arcuate shape to the absorbent article, as shown in FIG. 17(a). Alternatively, as shown in FIG. 17(b), a strip of elastic foam 47 may be laminated to the interior surface of the strip of material 55 that forms the cuff 25′, as previously discussed with respect to the embodiment shown in FIG. 6(a). As shown in FIG. 17(c), a cuff 25″ could be formed by wrapping a layer of fluid repellent porous foam material 46 around the strip of high loft material 55 to impart further cushioning for the cuff. The layer of foam 46 could itself be elasticized and applied to the absorbent article in tension, thereby eliminating the need for the elastic foam 15 to impart the arcuate shape.

Importantly, wings 19 are attached to the central portion 2 so that they cooperate with the cuffs 43 in use, as explained further below. In the preferred embodiment, the base 44 of each wing 19 is attached to a flange, as shown in FIG. 17. Thus, as shown in FIG. 17(b), a first strip of adhesive 34 is disposed between the portion 18 of cover layer 8 adjacent its longitudinal edge 33 and the portion 10 of the barrier layer 9 adjacent its longitudinal edge 33 and a second strip of adhesive is disposed between the opposite surface of the portion 10 of the barrier layer and the base 44 of the wing 19 so that the flange and wing base form a unitary structure. Alternatively, heat sealing could be used in place of adhesive strips 34. As a result this arrangement, the cuff 25 encloses the wing base 44, giving the absorbent article having cut and paste wings a more aesthetically pleasing appearance. More importantly, this method of attaching the wings to the absorbent article provides certain functional benefits, as described below.

The absorbent article 1 is generally applied to the crotch of a user's panty by placing the garment faceable side of the absorbent article against the inside surface of the panty crotch 27, as shown in FIG. 8. Pressure sensitive adhesive strips 21 are applied to the garment faceable side of the absorbent article to help maintain the absorbent article in place. As used herein, the term “pressure-sensitive adhesive” refers to any releasable adhesive or releasable tenacious means. Adhesive compositions suitable for sanitary napkins, include, for example, water-based pressure-sensitive adhesives such as acrylate adhesives, rapid setting thermoplastic “hot melt”, rubber adhesives, two-sided adhesive tape, and the like and combinations thereof.

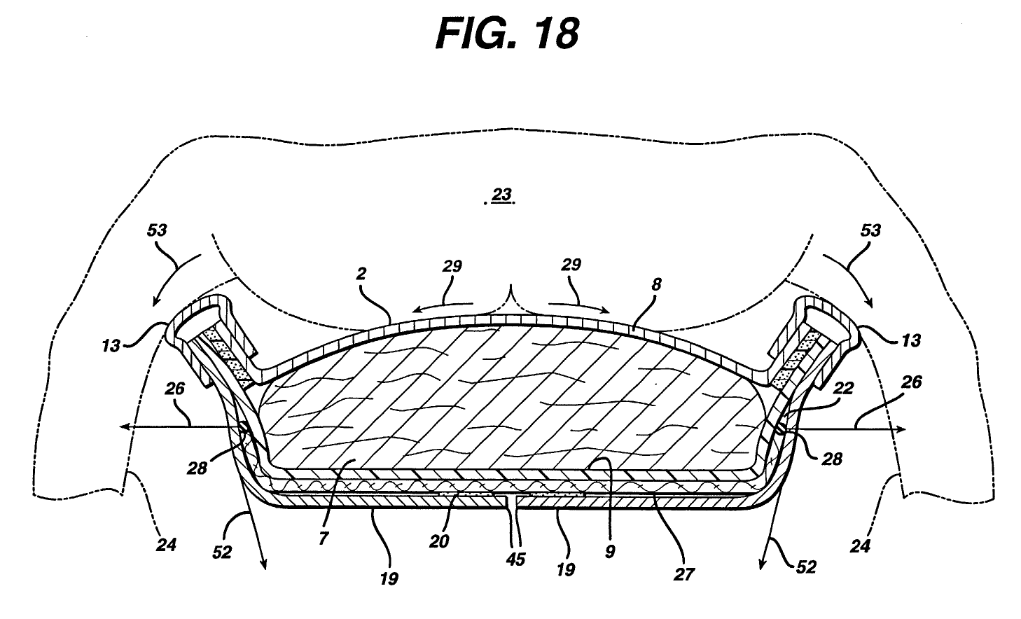

As is customary in the art, a paper release strip 56, which has been coated on one side, is applied to protect the adhesive strips 21 prior to use, as shown in FIGS. 4 and 5. The coating, which may be silicone, reduces the adherence to the adhesive of the coated side of the release strip. The release strip can be formed from any suitable sheet-like material which, when coated, adheres with sufficient tenacity to the adhesive to remain in place prior to use but which can be readily removed when the absorbent article is to be used. In use, the wings 19 are folded downward around the crotch 27 so that the edges of the wing tips 45 nearly abut each other and are secured to the underside of the crotch 27 via the adhesive 20, as shown in FIG. 18. As is known in the art, the wings 19 serve to stabilize the absorbent article and protect the panty crotch 27 from side leakage. However, unlike the wings heretofore known in the art, when the user pulls the wings 19 according to the current invention around the edges of the panty crotch 27 and attaches them thereto by adhesive 20, downward forces 52 are applied to the cuffs through the wing bases 44. These downward forces 52 impose moments 53 that tend to rotate the cuffs downward, as shown in FIG. 18. This downward rotation prevents the cuffs from folding inward over the body faceable surface 16 of the cover layer 8, thereby ensuring that effective placement of the cuffs is maintained. As previously discussed, the folding of the cuffs over the body faceable surface 16 reduces its effective area. Thus, according to the current invention, the wings 19 serve to place both the central portion 2 and the cuffs 43 into good contact with the body.

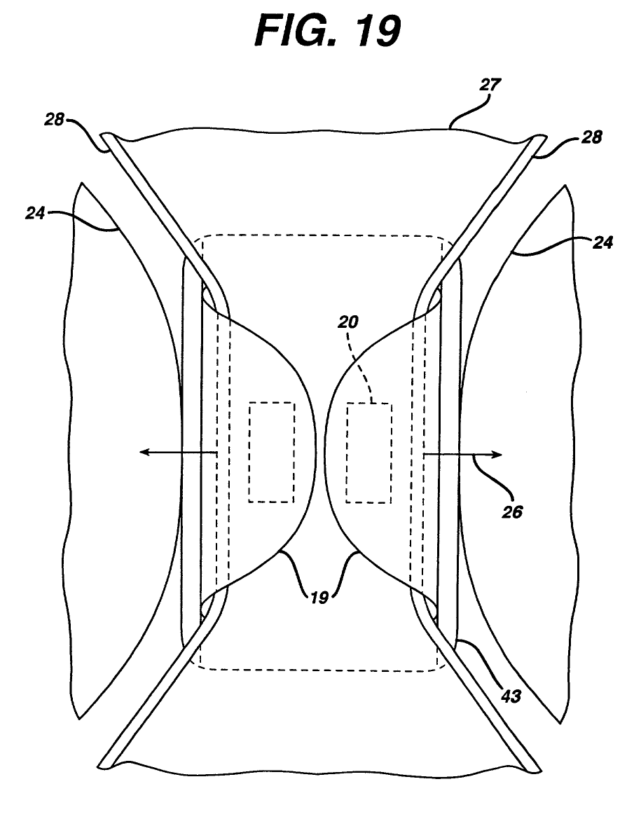

In the embodiment shown in FIG. 16, the intersection 49 of the wing base 44 and the barrier layer 9 forms a pocket 22 disposed at, or slightly inward of, the proximal end of the cuff 43. As shown in FIGS. 17(c) and 18, the pockets 22 serve to contain the edge portions 28 of the panty crotch 27. According to the current invention, certain advantages are obtained by attaching the wings 19 so that the distance F, shown in FIG. 16, between the pockets 22 is less than the width of the panty crotch 27 when the crotch is in its non-deformed state. Specifically, when the wings are attached to the panty crotch 27, as shown in FIGS. 18 and 19, the lateral compression of the crotch 27 causes the edges 28 to impart outward acting forces 26 on the wings 19. Since the wing base 44, barrier layer portion 10 and cover layer portion 18 are joined together so as to act in unison, the forces 26 are transmitted from the wings 19 to the cover layer 8, thereby placing the cover layer in tension, indicated by arrows 29 in FIG. 18. This tension causes the cover layer 8 to be thrust upward so as to ensure good contact with the perineum 23. The tension also serves to prevent permanent deformation of the article due to lateral compression from the user's thighs since the panty crotch edges 28 act as a spring to restore a laterally compressed central portion 2 to its non-deformed shape.

Another advantage of the cuff/wing arrangement shown in FIGS. 16 and 17 is that the pockets 22, and therefore, the panty crotch edges 28, are disposed below the base 12 of the cuff 43. As a result, the cuffs 43 extend a distance E, shown in FIG. 16, beyond the panty crotch edges. Unlike prior art attempts at sealing cuffs, the distance E is not limited to the thickness of the wing 19. Thus, the distal end 13 of the cuff makes sealing contact with the user's body regardless of the size or anatomical shape of the user or the panty crotch width.

The absorbent article shown in FIG. 16 discloses another important aspect of the current invention. As is well known, the cover layer 8 can be formed from an apertured polymeric film, such as RETICULON, APEX available from Chicopee Mills, Inc. of New York, N.Y., or DRI-WEAVE by The Proctor & Gamble Company of Cincinnati, Ohio. Although such materials have the advantage of imparting a dry feeling, some of these materials are uncomfortable against the body, tending to produce a hot and sticky feeling, as previously discussed.

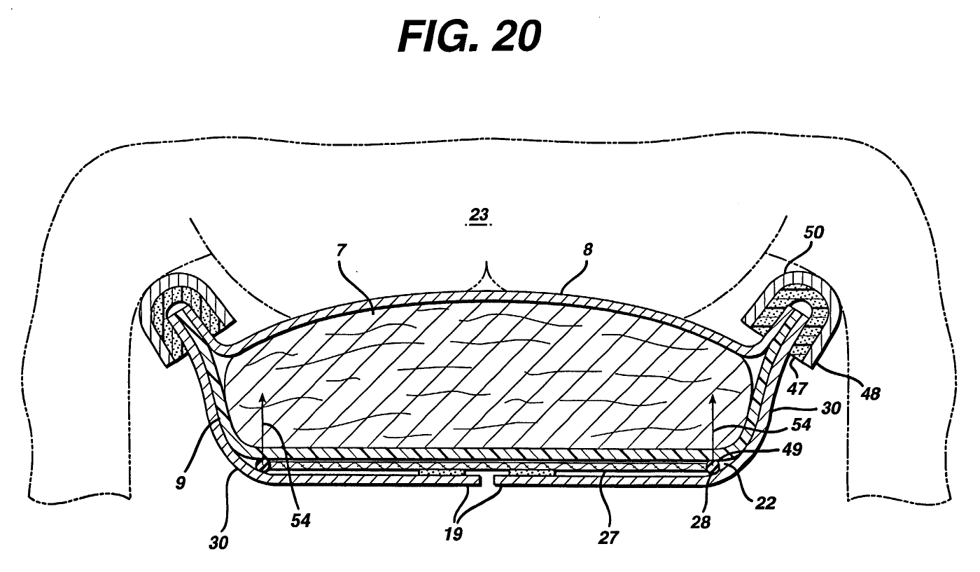

Consequently, as shown in FIG. 16, according to the current invention, an apertured polymeric film can be used only for the portion of the cover layer 8 directly over the absorbent core 7, where it is most beneficial. A second, more comfortable material can be used for the cuff 25 that bears against the body under pressure. Preferably this second material is a fibrous woven or nonwoven material, which, as is well known in the art, has a comfortable feeling against the body. Alternatively, a cuff 50 could be formed from a laminate of a layer 48 of a fibrous woven or nonwoven material and a layer of a porous, high loft elastic foam 47, as shown in FIG. 20.

According to the embodiment shown in FIG. 20, the wings 19 are attached to the barrier layer 9 so that, unlike the embodiment shown in FIG. 16, the initial intersection of the wings and the barrier layer occurs inward of the longitudinally extending sides 30 of the central portion 2, thereby similarly disposing the pockets 22. In this configuration, the elastic portions 28 of the panty crotch 27 impart forces 54 which act directly against the central portion 2 to enhance its contact with the perineum 23.

As the foregoing indicates, the method of the current invention affords great flexibility in the design of sanitary napkins, allowing the use of a wide range of cuff materials and allowing the cuffs to be attached to the absorbent article in various ways to achieve an optimum configuration. Moreover, although the invention has been explained with reference to a sanitary napkin, the invention is also suitable for use in other absorbent articles, such as incontinence pads and the like. As the various embodiments disclosed above indicate, the present invention may be embodied in many specific forms without departing from the spirit or essential attributes thereof and, accordingly, reference should be made to the appended claims, rather than to the foregoing specification, as indicating the scope of the invention.

What is claimed is:

1. An absorbent article for use in a perineal area of a user's body to absorb body fluid, the absorbent article comprising:
a) a fluid pervious first layer forming a top body faceable surface;
b) a fluid impervious second layer forming a bottom garment faceable surface opposite the body faceable surface;
c) an absorbent core positioned between the first layer and the second layer;
d) right and left cuffs which extend along a substantial portion of opposite right and left lateral sides of the absorbent article, respectively, the cuffs being located in at least a central portion of the absorbent article, each cuff having a base portion and a distal end, each cuff comprising an inner layer comprising a strip of resilient, highloft, fluid permeable material, and an outer layer comprising a flexible, fluid repellent porous material, said outer layer substantially covering said inner layer, and wherein the right and left cuffs are attached along their respective base portions to the right and left lateral sides of the absorbent article, respectively, such that the distal ends of the cuffs extend outward from the right and left lateral sides of the absorbent article, respectively.

2. The absorbent article according to claim 1, wherein the absorbent article has a longitudinal dimension and a transverse dimension, the longitudinal dimension corresponding to the right and left lateral sides of the absorbent article; the fluid pervious first layer having a right and left edge, and the fluid impervious second layer having a right and left edge, and wherein the fluid pervious first layer is joined to the fluid impervious second layer adjacent to their respective right and left edges so as to form right and left flanges, wherein the right and left flanges are adjacent the right and left lateral sides of the absorbent article, respectively, and wherein the right and left cuffs are adhered to at least a portion of the right and left flanges, respectively.

3. The absorbent article according to claim 1, wherein the strip of resilient, highloft, fluid permeable material is formed from fibers selected from the group consisting of polyester fibers, polyethylene/polyester bicomponent fibers, polyethylene/polypropylene bicomponent fibers, polypropylene/polyester bicomponent fibers, high melting/low melting polyester bicomponent fibers, air laid pulp, pulp-fiber blends, and combinations thereof.

4. The absorbent article according to claim 1, wherein the fluid repellant porous material is selected from the group consisting of apertured polymeric films, nonwoven fabrics and woven fabrics.

5. The absorbent article according to claim 2, wherein the flexible, fluid repellent porous material of each cuff is folded over the respective flange to form a cavity at the distal end of its respective cuff.

6. The absorbent article according to claim 12, wherein the cavity forms a compliant portion at the distal end of each respective cuff that deforms in response to compressive forces imparted by the user's body.

7. The absorbent article according to claim 1, wherein the absorbent core has an hourglass shape with enlarged end portions separated by a narrower center portion and wherein the center portion is thicker than the end portions.

8. The absorbent article according to claim 20, wherein the center portion further comprises sphagnum moss.
